(19) United States
(12) Patent Application Publication
(10) Pub. No.: US 2026/0226080 A1
(43) Pub. Date: Aug. 6, 2026

(54) SULFONATED-MODIFIED METAL-ORGANIC FRAMEWORK MATERIAL, PREPARATION METHOD THEREFOR, AND APPLICATION THEREOF IN YIELD INCREASE OF ARYL GLYCOSIDE COMPOUNDS

(71) Applicant: Nanjing Normal University, Jiangsu (CN)

(72) Inventors: Xing ZHANG, Jiangsu (CN); Jie Zheng, Jiangsu (CN); Meng Qiao, Jiangsu (CN); He Huang, Jiangsu (CN)

(73) Assignee: Nanjing Normal University, Jiangsu (CN)

(21) Appl. No.: 19/650,265

(22) Filed: Apr. 16, 2026

Related U.S. Application Data
(63) Continuation of application No. PCT/CN2025/132035, filed on Nov. 3, 2025.

(30) Foreign Application Priority Data
Dec. 27, 2024 (CN) 202411950494.X

(51) Int. Cl. C07F 7/00; C01B 39/00; C12N 1/145; C12P 7/22; C12P 19/60; C12R 1/645

(52) U.S. Cl. CPC C07F 7/00; C01B 39/00; C12N 1/145; C12P 7/22; C12P 19/60; C12R 2001/645

(57) ABSTRACT

A sulfonated-modified metal-organic framework material, a preparation method therefor, and an application thereof in yield increase of aryl glycoside compounds are provided, and the sulfonated-modified metal-organic framework material is prepared through a reaction with 1,2-ethanedisulfonic acid for sulfonation modification where a zirconium-based MOF material Zr-BTB serves as a precursor. The sZr-BTB material prepared in the present disclosure is immobilized on a surface of Yarrowia lipolytica through physical adsorption to form a dense protective film, such that intracellular retention of relatively larger-molecule glycosidases prevents secretion but the efflux of small-molecule aryl glycoside compounds is not affected, thereby preventing the decomposition of a biosynthesized main product. Moreover, the present disclosure maintains stable adsorption of Yarrowia lipolytica under strongly acidic conditions, thereby facilitating yield increase of aryl glycoside compounds by fermentation, overcoming the obstacles of non-model microorganisms, and promoting the low-cost industrial biosynthetic production.

### CROSS-REFERENCE TO RELATED APPLICATION

The present application is a continuation of PCT application serial No. PCT/CN2025/132035, filed on Nov. 3, 2025, which claims the priority benefit of China application no. 202411950494.X filed on Dec. 27, 2024. The entirety of each of the above mentioned patent applications is incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Dec. 26, 2024, is named 165420-US-sequence and is 6,982 bytes in size.

### BACKGROUND

### Technical Field

The present disclosure belongs to the field of biochemical engineering, and particularly relates to a sulfonated-modified metal-organic framework material, a preparation method therefor, and an application thereof in yield increase of aryl glycoside compounds.

### Description of Related Art

Microbial strains as the core of modern industrial biotechnology play an increasingly important role in the industrial field. The catalytic efficiency achieved through the technology of microbial catalytic reactions based on metabolism is many times higher than that of chemical catalytic reactions. Additionally, biocatalytic reactions may occur at room temperature with high catalytic specificity, and have the advantages of low energy consumption and high safety. Model microorganisms such as Escherichia coli and Saccharomyces cerevisiae have long been regarded as the ideal microbial chassis. However, in recent years, non-model microorganisms have become the chassis for synthesis of many natural products due to technological breakthroughs. These non-model microorganisms have special physiological and metabolic characteristics and provide abundant genetic resources, thereby facilitating the development of new products. Yarrowia lipolytica as an unconventional hemiascomycetous yeast is considered an excellent natural synthesis host. Yarrowia lipolytica is used for the synthesis of aryl glycoside compounds with glucose as a substrate. However, non-negligible defects exist in production of aryl glycoside compounds by fermentation of Yarrowia lipolytica. During the growth of Yarrowia lipolytica, by-products such as organic acids are typically produced, causing a pH value of a culture medium to drop to an extremely acidic level (pH 2-3). In a strong acid environment, Yarrowia lipolytica easily loses activity. Additionally, during the fermentation of Yarrowia lipolytica for production, hydrolases such as glycosidases and small-molecule products are secreted extracellularly simultaneously, and glycosidases hydrolyze aryl glycoside compounds into by-products, resulting in a decrease in a yield of a main product. These problems hinder the industrial production of aryl glycoside compounds using Yarrowia lipolytica.

### SUMMARY

Invention objective: to address the deficiencies in production of aryl glycoside compounds by biological fermentation of Yarrowia lipolytica, the present disclosure provides a sulfonated-modified metal-organic framework material; the sulfonated-modified metal-organic framework material prepared in the present disclosure is a novel zirconium-based metal-organic framework (MOF) material, i.e., sulfonated-modified Zr-BTB (sZr-BTB), used for immobilizing Yarrowia lipolytica. In the process of adsorbing Yarrowia lipolytica, sZr-BTB inhibits the secretion of hydrolases such as glycosidases during fermentation of Yarrowia lipolytica, thereby reducing product degradation; and additionally, sZr-BTB is continuously and stably adsorbed to a surface of Yarrowia lipolytica under acidic conditions, thereby providing long-term protection for Yarrowia lipolytica under strongly acidic conditions, and effectively solving the problems of low yield of aryl glycoside compounds produced by fermentation of Yarrowia lipolytica and low yeast activity.

The present disclosure further provides a preparation method for the sulfonated-modified metal-organic framework material and an application thereof.

Technical solution: to achieve the above objective, the sulfonated-modified metal-organic framework material of the present disclosure is provided, and the sulfonated-modified metal-organic framework material is prepared through a reaction with 1,2-ethanedisulfonic acid for sulfonation modification where a zirconium (Zr)-based MOF material Zr-BTB serves as a precursor.

The preparation method for the sulfonated-modified Zr-BTB material of the present disclosure includes the following steps:
(1) preparing a metal-organic framework material Zr-BTB: dissolving 1,3,5-tris(4-carboxyphenyl)benzene (H₃BTB), benzoic acid (BA), and zirconium tetrachloride (ZrCl₄) in an organic solvent;
(2) adding deionized water to the organic solvent, performing ultrasonic mixing for a high-temperature reaction, washing a resulting mixture after cooling, and centrifuging and drying to obtain the Zr-BTB material as a precursor; and
(3) adding 1,2-ethanedisulfonic acid to the prepared Zr-BTB material as the precursor, centrifuging a resulting mixture after a high-temperature reaction, collecting a precipitate and washing with deionized water to obtain a sulfonated-modified Zr-BTB material, i.e., the sulfonated-modified metal-organic framework material.

In the step (1), a mass ratio of H₃BTB to BA to ZrCl₄ is 10-13:440-460:9-12.

In the step (2), the high-temperature reaction is performed at 100-130° C. for 20-24 h, and the centrifuging is performed at 8000-12000 rpm for 5-10 min.

In the step (3), 1,2-ethanedisulfonic acid is dissolved in the organic solvent, a mass ratio of 1,2-ethanedisulfonic acid to the Zr-BTB material as the precursor is 1:1-6:1, the high-temperature reaction is performed at 90-120° C. for 15-20 h, and the centrifuging is performed at 8000-12000 rpm for 5-10 min.

Preferably, the preparation method for the sulfonated-modified Zr-BTB material includes the following steps:
(1) dissolving H₃BTB, BA, and ZrCl₄ in dimethylformamide (DMF);
(2) adding a certain amount of deionized water to the above solution, performing ultrasonic mixing for a high-temperature reaction in a reaction kettle, and washing with DMF and acetone after cooling, and centrifuging and drying to obtain the Zr-BTB material as a precursor; and
(3) adding the Zr-BTB material as the precursor into a 1,2-ethanedisulfonic acid solution for a high-temperature reaction, and then centrifuging a resulting mixture to collect the sulfonated-modified Zr-BTB material.

An amount of DMF in the step (1) is 2-4 mL; and amounts of H₃BTB, BA, and ZrCl₄ are 10-13 mg, 440-460 mg, and 9-12 mg respectively.

A volume of deionized water in the step (2) is 0.5-1 mL.

Preferably, after the Zr-BTB material as the precursor is washed with DMF and acetone, the Zr-BTB material is further washed once with 0.1 M hydrochloric acid, and then the hydrochloric acid is removed with deionized water.

An amount of the Zr-BTB material in the step (3) is 15-25 mg, an amount of 1,2-ethanedisulfonic acid is 10-80 mg, and a mass ratio of the Zr-BTB material as the precursor to 1,2-ethanedisulfonic acid is 1:1-1:4.

More preferably, the preparation method for the sulfonated-modified Zr-BTB material includes the following steps:
(1) Dissolving 10-13 mg of H₃BTB, 440-460 mg of BA, and 9-12 mg of ZrCl₄ in 2-4 mL of DMF.
(2) After centrifuging the above solution to remove a supernatant, adding a 0.1 M hydrochloric acid solution, performing a reaction of the solvent in a reaction kettle at 100-130° C. for 20-24 h, washing with DMF for 1-3 times, washing with acetone for 1-3 times, washing with 0.1 M hydrochloric acid once, and washing with deionized water for 1-3 times after cooling, centrifuging at 8000-12000 rpm for 5-10 min, and then drying to obtain the Zr-BTB material.
(3) Dissolving 10-200 mg of 1,2-ethanedisulfonic acid in 2-5 mL of DMF, then adding a resulting solution to 15-25 mg of the Zr-BTB material as the precursor, performing a reaction of a resulting mixture at 90-120° C. for 15-20 h, and then centrifuging at 8000-12000 rpm for 5-10 min to collect the sulfonated Zr-BTB material.

The present disclosure provides an application of the sulfonated-modified metal-organic framework material in production of aryl glycoside compounds by fermentation of Yarrowia lipolytica.

The sulfonated-modified metal-organic framework (sZr-BTB) material of the present disclosure is combined with Yarrowia lipolytica cells through electrostatic adsorption, and the immobilized Yarrowia lipolytica inhibits the secretion of glycosidases during fermentation, thereby achieving an increase in production of aryl glycoside compounds by microbial fermentation. Additionally, sZr-BTB stably adsorbs Yarrowia lipolytica under strongly acidic conditions, thereby protecting Yarrowia lipolytica and improving the catalytic efficiency.

A process of the application includes the following steps:
(1) inoculating Yarrowia lipolytica into a seed culture medium for seed culture;
(2) inoculating a seed culture solution into a fermentation medium for fermentation culture; and
(3) adding the sulfonated-modified metal-organic framework material into a fermentation broth to achieve the immobilization of yeast cells in a logarithmic growth phase, and continuing fermentation to achieve the yield increase of a target product, i.e., aryl glycoside compounds.

In the step (1), Yarrowia lipolytica is cultured in a yeast extract peptone dextrose (YPD) seed culture medium for 24-36 h; and in the step (2), culture is further performed in the fermentation medium for 24-36 h.

In the step (3), the sulfonated-modified metal-organic framework material is added to the fermentation broth, specifically, 0.5-1.5 mg of the sZr-BTB material is added per 20-30 mL of the fermentation broth, immobilization starts in the logarithmic growth phase after material addition, and fermentation is continued for 100-120 h to achieve the yield increase of the target product, i.e., aryl glycoside compounds.

The aryl glycoside compounds include one or more of gastrodin, arbutin, resveratrol, naringin, scutellarin, and isovitexin.

Preferably, the application includes the following steps:
(1) Inoculating a glycerol-preserved strain into 3 mL of a YPD seed culture medium, and performing seed culture for 24-36 h under certain temperature and rotation speed conditions.
(2) Inoculating 1.5 mL of a seed culture solution into 30 mL of a fermentation medium, and performing culture for 24-36 h under certain temperature and rotation speed conditions.
(3) Adding the sulfonated-modified metal-organic framework material into a fermentation broth, continuing fermentation for 120 h, sampling every 24 h, freezing the sampled fermentation broth at −20° C. in a refrigerator, and measuring a yield of gastrodin.

The strain in the step (1) is Yarrowia lipolytica. The YPD seed culture medium is composed of 10 g/L yeast extract, 20 g/L peptone, and 80 g/L anhydrous glucose. Culture is performed at a temperature of 30° C. and a rotation speed of 220-250 rpm.

The fermentation medium in the step (2) is composed of 10 g/L yeast extract, 20 g/L peptone, 80 g/L anhydrous glucose, and 5-10 mg/L trace metal elements (4.0 g/LFeSO₄·7H₂O, 4.0 g/L CaCl₂, 1.0 g/L MgCl₂). Culture is performed at a temperature of 30° C. and a rotation speed of 220-250 rpm.

In the step (3), 0.5-1.5 mg of the sulfonated-modified Zr-BTB material is added per 30 mL of the fermentation broth.

The present disclosure provides a method for immobilizing Yarrowia lipolytica by using a sulfonated-modified metal-organic framework material (sZr-BTB). Compared with free Yarrowia lipolytica, immobilized Yarrowia lipolytica is less susceptible to external influences during growth and metabolism, thereby maintaining yeast activity. First, the immobilized Yarrowia lipolytica is easily recovered during fermentation or biotransformation, thereby achieving microbial reuse. The sZr-BTB prepared by the present disclosure is a material with a high specific surface area and porosity, and the high specific surface area and highly ordered pore structure endow sZr-BTB with strong adsorption capacity and carrier capacity. Additionally, sZr-BTB has good biocompatibility without adverse effects on the growth, metabolism, and activity of cells. The sZr-BTB provided by the present disclosure coats Yarrowia lipolytica through physical adsorption to form a protective film on a yeast surface, such that intracellular retention of glycosidases prevents secretion but the efflux of small-molecule products is allowed, thereby preventing the decomposition of biosynthesized aryl glycoside compounds. Additionally, sulfonated groups on the sZr-BTB material compete with groups on the Yarrowia lipolytica cell surface for hydrogen ions in a fermentation broth with strong acidity, thereby ensuring that the Zr-BTB material permanently coats Yarrowia lipolytica and protects yeast cells.

In summary, the method for yeast immobilization using sZr-BTB in the present disclosure overcomes some major defects of the non-model microorganism Yarrowia lipolytica. The method not only effectively prevents the loss of yeast activity, but also enhances adsorption and carrier functions through the high specific surface area and pore structure. Sulfonated groups of sZr-BTB protect yeast cells under acidic conditions, prevent the extracellular secretion of glycosidases, and prevent degradation of aryl glycoside compounds. The method of the present disclosure significantly enhances the stability and efficiency of Yarrowia lipolytica in industrial production, showing broader prospects in biotechnology applications.

During the fermentation of Yarrowia lipolytica, glycosidases and products are secreted extracellularly, and glycosidases decompose a part of the biosynthesized aryl glycoside compounds into by-products, resulting in a low yield of a main product. Secondly, normal Yarrowia lipolytica tends to produce by-products such as organic acids during fermentation, thereby leading to a significant decrease in pH values of a fermentation broth and ultimately possible yeast death. To solve these problems, the sZr-BTB material is provided in the present disclosure, and the Zr-BTB material is immobilized on a surface of Yarrowia lipolytica through physical adsorption to form a dense protective film, such that intracellular retention of relatively larger-molecule glycosidases prevents secretion but the efflux of small-molecule aryl glycoside compounds is not affected, thereby preventing the decomposition of a biosynthesized main product. Additionally, sulfonic acid groups on the sZr-BTB material provided in the present disclosure compete with anionic groups on a yeast surface for active hydrogen ions in a fermentation broth, thereby enhancing the capacity of adsorption of Yarrowia lipolytica by the Zr-BTB material under acidic conditions, and the protective film further improves the stability of Yarrowia lipolytica cells and also the catalytic efficiency and reusability. The sulfonated-modified Zr-BTB (sZr-BTB) prepared in the present disclosure maintains stable adsorption of Yarrowia lipolytica under strongly acidic conditions, and adsorption and coating prevent the extracellular secretion of glycosidases and the like from Yarrowia lipolytica, thereby facilitating yield increase of aryl glycoside compounds by fermentation, overcoming the obstacles of non-model microorganisms, and promoting the low-cost industrial biosynthetic production.

The present disclosure first provides a method for preparing a sulfonated-modified zirconium-based material sZr-BTB, and applies the Zr-BTB material to the fermentation of Yarrowia lipolytica to achieve the yield increase of aryl glycoside compounds. The sulfonated-modified Zr-BTB material designed in the present disclosure coats Yarrowia lipolytica like a membrane, thereby restricting the secretion of yeast glycosidases while ensuring the efflux of substrates. Moreover, since Yarrowia lipolytica is coated by the Zr-BTB material, Yarrowia lipolytica maintains a certain activity in a fermentation broth with extremely strong acidity, thereby increasing the production of aryl glycoside compounds by fermentation of Yarrowia lipolytica and enhancing Yarrowia lipolytica activity in harsh environments.

Currently, in the prior art, secretion of glycosidases cannot be controlled during the fermentation of Yarrowia lipolytica, resulting in the decomposition of the main product aryl glycoside compounds by glycosidases. Furthermore, an acidity of a fermentation broth of yeast is approximately pH 2-3, and yeast activity decreases in a harsh environment. The sZr-BTB material provided in the present disclosure coats Yarrowia lipolytica to prevent the secretion of glycosidases, and protects Yarrowia lipolytica in an acidic fermentation broth and maintains high activity.

Compared with the prior art, the present disclosure has the following advantages and beneficial effects:

1. The Zr-BTB material is used in the present disclosure, Zirconium metal is considered to be low-toxic or even non-toxic, and compared with other metal-organic framework materials, Zr-BTB is a two-dimensional metal-organic framework (2D-MOF) material with a larger planar size, a smaller thickness, and higher water stability, indicating that Zr-BTB easily embeds yeast cells, with a structure not easily damaged.

2. The present disclosure provides a method for producing aryl glycoside compounds by microbial fermentation of Yarrowia lipolytica, and the method has the advantages of high yield, easy scale-up culture and short culture cycle, and is more suitable for the industrial production of aryl glycoside compounds.

3. The present disclosure combines MOF material nanosheets with Yarrowia lipolytica through physical adsorption, and compared with free Yarrowia lipolytica, the immobilized Yarrowia lipolytica exhibits more stability during metabolism. Additionally, physical adsorption between Yarrowia lipolytica and the Zr-BTB material enables easy separation of a product from Yarrowia lipolytica, and the subsequent separation and purification process is simplified.

4. A lot of organic acids are produced during fermentation of Yarrowia lipolytica, and compared with conventional metal-organic framework materials, Zr-BTB exhibits more stability in an acidic environment, such that the Zr-BTB material maintains long-term structural integrity under acidic conditions without disintegration.

5. The present disclosure provides a method for modifying Zr-BTB after synthesis by using 1,2-ethanedisulfonic acid, one sulfonic acid group of 1,2-ethanedisulfonic acid coordinates with zirconium metal on Zr-BTB, and the other sulfonic acid group is configured to compete with groups on a yeast cell surface for hydrogen ions under acidic conditions, thereby ensuring that sZr-BTB maintains stability of adsorbing Yarrowia lipolytica for a long time under strongly acidic conditions.

6. Glycosidases produced during the fermentation of Yarrowia lipolytica decompose the main product aryl glycoside compounds, and the sulfonated-modified Zr-BTB material as a 2D-MOF material coats a surface of Yarrowia lipolytica, thereby inhibiting the secretion of hydrolases such as glycosidases into a fermentation broth without affecting the efflux of small-molecule aryl glycoside compounds, thereby further preventing the decomposition of a biosynthesized main product and preventing the decomposition of products by enzymes. The immobilization effect increases the yield of the product aryl glycoside compounds, and since Yarrowia lipolytica is coated by the sulfonated-modified Zr-BTB material, Yarrowia lipolytica maintains excellent activity in a fermentation broth with extremely strong acidity or any other harsh fermentation environment. Furthermore, the Zr-BTB material prepared in the present disclosure has a good protective effect on Yarrowia lipolytica under high temperature conditions, and enhances the stability of Yarrowia lipolytica, and a yield of gastrodin at a higher fermentation temperature such as 45° C. is much higher than that of free Yarrowia lipolytica. 7. The preparation method of the present disclosure is simple and conveniently implemented, and raw materials are easily obtained, such that large-scale production and application are effectively achieved, obstacles of non-model microorganisms are overcome, and the low-cost industrial biosynthetic production is promoted.

### BRIEF DESCRIPTION OF THE DRAWINGS

FIG. 1 illustrates scanning electron microscope comparison of immobilized Yarrowia lipolytica and free Yarrowia lipolytica provided in Example 1 and Comparative Example 1.

FIG. 2 is a high performance liquid chromatography spectrum of gastrodin and p-hydroxybenzyl alcohol in Example 1.

FIG. 3 illustrates yield comparison of gastrodin in Example 1 and Comparative Example 1.

FIG. 4 illustrates ratios of gastrodin to p-hydroxybenzyl alcohol in Example 1 and Comparative Example 1.

FIG. 5 is a high performance liquid chromatography spectrum of gastrodin and p-hydroxybenzyl alcohol in Comparative Example 1.

FIG. 6 illustrates yields of gastrodin produced by fermentation of Yarrowia lipolytica immobilized by sZr-BTB with different ratios of zirconium tetrachloride in Example 2.

FIG. 7 illustrates yields of gastrodin produced by fermentation of Yarrowia lipolytica immobilized by sZr-BTB with different ratios of 1,3,5-tris(4-carboxyphenyl)benzene in Example 3.

FIG. 8 illustrates yields of gastrodin produced by fermentation of Yarrowia lipolytica immobilized by sZr-BTB with different ratios of benzoic acid in Example 4.

FIG. 9 illustrates yields of gastrodin produced by fermentation of Yarrowia lipolytica immobilized by sZr-BTB with different ratios of 1,2-ethanedisulfonic acid in Example 5.

FIG. 10 illustrates yields of gastrodin produced by fermentation of Yarrowia lipolytica immobilized by different masses of sZr-BTB in Example 6.

FIG. 11 illustrates yields of gastrodin produced by fermentation of immobilized Yarrowia lipolytica at different temperatures in Example 7.

FIG. 12 illustrates yields of gastrodin produced by fermentation of immobilized Yarrowia lipolytica in different organic solvents in Example 8.

### DESCRIPTION OF THE EMBODIMENTS

The present disclosure will be further described with reference to the accompanying drawings and the embodiments.

Unless otherwise specified, the materials, reagents and so forth used in the embodiments of the present disclosure can be obtained commercially. If specific conditions of experimental methods are not specified in the following embodiments, conventional conditions or conditions recommended by the manufacturer shall prevail.

In the following embodiments, 1,3,5-tris(4-carboxyphenyl)benzene, benzoic acid, zirconium tetrachloride, dimethylformamide, and 1,2-ethanedisulfonic acid were purchased from Macklin Biochemical Co., Ltd.

As used herein, the term “MOF” refers to a metal-organic framework.

As used herein, the term “YPD” refers to a yeast extract peptone dextrose medium.

As used herein, the term “BA” refers to benzoic acid.

As used herein, the term “DMF” refers to dimethylformamide.

As used herein, the term “sZr-BTB” refers to sulfonated-modified Zr-BTB.

A sugar-containing fermentation medium: 10 g/L yeast extract, 20 g/L peptone, 80 g/L anhydrous glucose, 5 mg/L trace metal elements (4.0 g/L FeSO₄·7H₂O, 4.0 g/L CaCl₂, 1.0 g/L MgCl₂).

Gastrodin-producing Yarrowia lipolytica used in the embodiments of the present disclosure is a YliGT6 strain provided by Nanjing Normal University, with details in a method for yield increase of gastrodin by using recombinant Yarrowia lipolytica disclosed in Chinese patent application No. CN 117844844 A.

A starting strain of arbutin-producing Yarrowia lipolytica is Y. lipolytica PO1f (ATCC no. MYA-2613™).

The arbutin-producing Yarrowia lipolytica is obtained by engineering transformation in a laboratory, and main operation steps include: S1. constructing a heterologous expression plasmid of Yarrowia lipolytica; and S2. preparing a seed culture solution and purifying to obtain the arbutin-producing Yarrowia lipolytica.

### S1 Includes the Following Steps:

Heterologously expressing an AS gene (SEQ ID NO.1), a UbiC gene (SEQ ID NO.2), and an MNX1 gene (SEQ ID NO.3). Exogenous genes AS, UbiC and MNX1 are used for the synthesis of arbutin. Selecting a YaliBrick plasmid pYLXP′ (purchased from Hangzhou Baosai Biotechnology Co., Ltd.) as a backbone, designing homology arms while performing PCR amplification on both a plasmid backbone and target fragments, and then performing fragment splicing through a Gibson assembly method to construct heterologous gene expression plasmids pYLXP′-AS, pYLXP′-UbiC, and pYLXP′-MNX1 respectively.

Splicing pYLXP′-AS and pYLXP′-MNX1 plasmids digested with restriction endonucleases BlnI and SalI through T4 ligase to obtain a plasmid pYLXP′-AS-MNX1, and performing enzymatic digestion and ligation in the same way to obtain a plasmid pYLXP′-AS-MNX1-UbiC. Transforming the constructed plasmids into the starting strain Y. lipolytica PO1f (polg Δura3), and spreading a resulting transformation mixture onto a YPD plate (g/L) containing 5-fluoroorotic acid (50 mg/L) (10 g/L yeast extract, 20 g/L peptone, 20 g/L glucose, and 15 g/L agar powder) for culture, performing PCR verification by using 2×TOROBlue® Flash KOD DyeMix, and screening a successfully transformed single clone for a next experimental operation.

### S2. Preparing a Seed Culture Solution and Obtaining a Strain:

Randomly selecting a successfully screened transformant from the plate cultured overnight, inoculating the transformant into 3 mL of a YPD culture solution (including 10 g/L yeast extract, 20 g/L peptone, and 25 g/L glucose), and performing shaking culture at 220 rpm and 30° C. for 120 h.

After 120 h of shaking culture, centrifuging at 12000 rpm for 10 min to collect a supernatant, filtering the supernatant through a 0.22 m aqueous filter membrane with a syringe to prepare a sample, then detecting the presence of arbutin and measuring arbutin content using a Liquid Chromatograph Mass Spectrometer, where a mobile phase ratio of water (containing 0.05% trifluoroacetic acid) to methanol is (90:10), a flow rate is 0.7 mL/min, a column temperature is set to 25° C., a detection wavelength is 282 nm, and an injection volume is set to 20 μL.

Plotting a standard curve according to quantitative analysis results, and determining that a clone achieves a high yield of arbutin for subsequent fermentation.

Streaking an obtained high-yield arbutin-producing yeast transformant on a YPD plate, picking a single colony with a sterile toothpick and inoculating into a YPD medium, and performing shaking culture at 220 rpm and 30° C. for 24 h.

Inoculating 500 μL of a seed culture solution into 30 ml of a YPD fermentation broth (including 10 g/L yeast extract, 20 g/L peptone, and 50 g/L glucose), and performing continuous fermentation culture at 30° C. and 220 rpm for 120 h.

Sampling every 24 h to determine OD₆₀₀, centrifuging a sample at 12000 rpm for 10 min to collect a supernatant, filtering the supernatant through a 0.22 m aqueous filter membrane to prepare a target sample for high performance liquid chromatography (HPLC) detection, and accurately determine an arbutin yield, where the strain as an engineering strain is used for subsequent immobilization operations.

### Example 1

In this example, the capability of sZr-BTB to promote the production of aryl glycoside compounds by fermentation of Yarrowia lipolytica was explored, a gastrodin-producing Yarrowia lipolytica strain was adopted in this example, and preparation steps of sZr-BTB were as follows:
(1) 10 mg of zirconium tetrachloride, 12 mg of 1,3,5-tris(4-carboxyphenyl)benzene, and 450 mg of BA were dissolved in 3 mL of DMF, and then 0.5 mL of deionized water was added for ultrasonic dissolution. Then a resulting mixture was stirred in a water bath at 120° C. for 24 h, after cooling, and the mixture was washed with DMF for 3 times, with acetone for 3 times, with 0.1 M hydrochloric acid for once, and with deionized water for 2 times, then centrifuged at 8000 rpm for 5 min, and dried to obtain solid Zr-BTB.
(2) 80 mg of 1,2-ethanedisulfonic acid was added to 4 mL of DMF for ultrasonic dissolution, a resulting solution was added to 40 mg of Zr-BTB prepared above, after mixing uniformly, a resulting mixture was stirred in a water bath at 100° C. and 150 rpm for 20 h, and after cooling, the mixture was washed with DMF for 3 times and with acetone for 3 times, centrifuged at 8000 rpm for 5 min, and dried to obtain an sZr-BTB material.

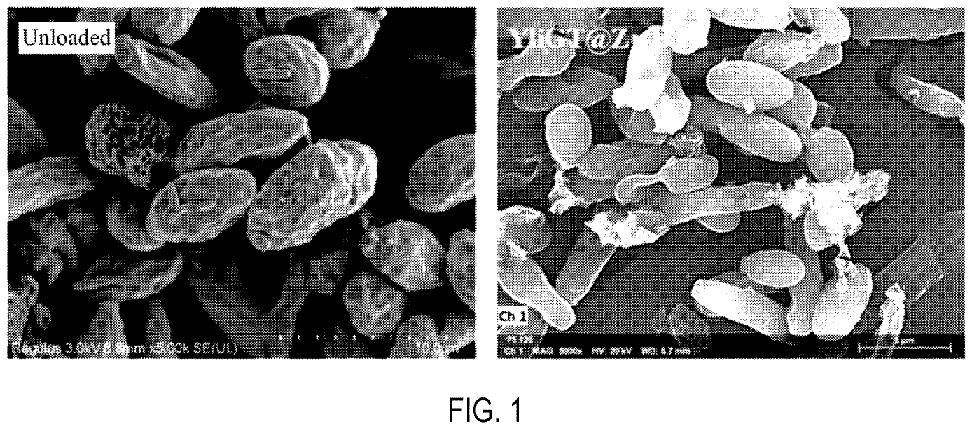

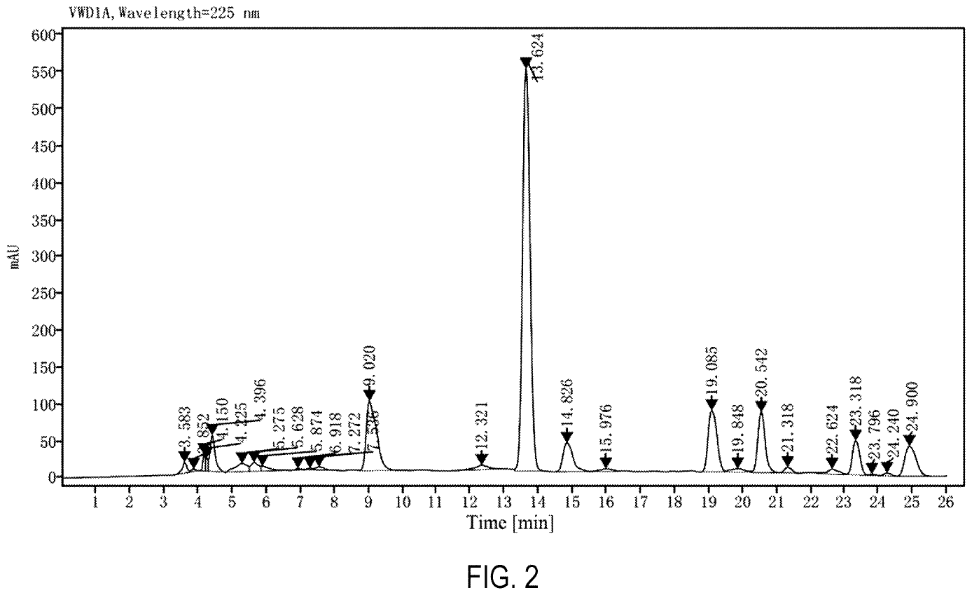

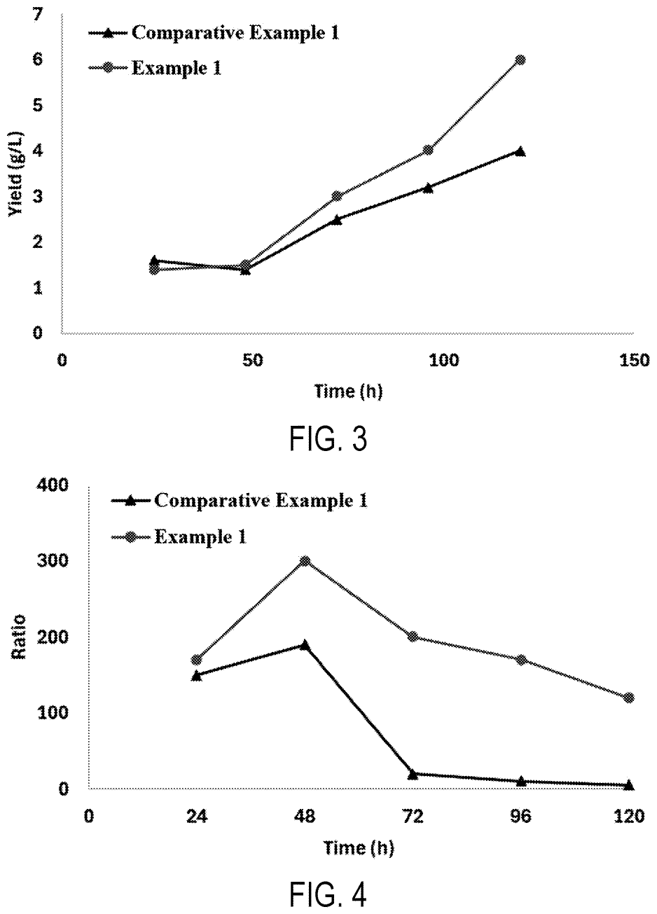

The sZr-BTB-immobilized gastrodin-producing Yarrowia lipolytica (YliGT6) is prepared as follows:
(1) YliGT6 in a glycerol tube was inoculated into 3 mL of a YPD seed culture medium, and after mixing uniformly, a resulting mixture was cultured with shaking in a constant temperature shaker for shaking fermentation at 30° C. and 250 rpm for 24 h.
(2) 1.5 mL of activated Yarrowia lipolytica was inoculated into 30 mL of a fresh sugar-containing fermentation medium, and after mixing uniformly, a resulting mixture was cultured with shaking in a constant temperature shaker for shaking fermentation at 30° C. and 250 rpm for 24 h until OD₆₀₀ was approximately 0.7.
(3) 1 mg of sZr-BTB was added to 30 mL of a fermentation broth, and a resulting mixture was fermented in a constant temperature shaker for shaking fermentation at 30° C. and 250 rpm for 120 h. Samples of the fermentation broth were taken every 24 h, and frozen at −20° C. in a refrigerator for preservation. A scanning electron microscope image of loaded Yarrowia lipolytica is shown in FIG. 1.
(4) The samples in the step (3) were detected by liquid chromatography. A SinoChrom ODS-BP column was used for high performance liquid chromatography, with a mobile phase of 10% (methanol/water), and a flow rate of 0.7-1 mL/min. A wavelength of an ultraviolet detector was set to 225 nm. Peaks of a main product gastrodin and a by-product p-hydroxybenzyl alcohol detected by liquid chromatography are shown in FIG. 2, a peak of gastrodin appeared at approximately 13.8 min with a peak area of 13.624, and a peak of p-hydroxybenzyl alcohol appeared at approximately 19 min with a peak area of 19.085. Yields of gastrodin inferred from the peaks are shown in FIG. 3. Ratios of gastrodin to p-hydroxybenzyl alcohol are shown in FIG. 4. It can be seen from the results that the yield of gastrodin increased gradually over time, but the yield of gastrodin decreased after 48 h due to production of glycosidases.

### Comparative Example 1

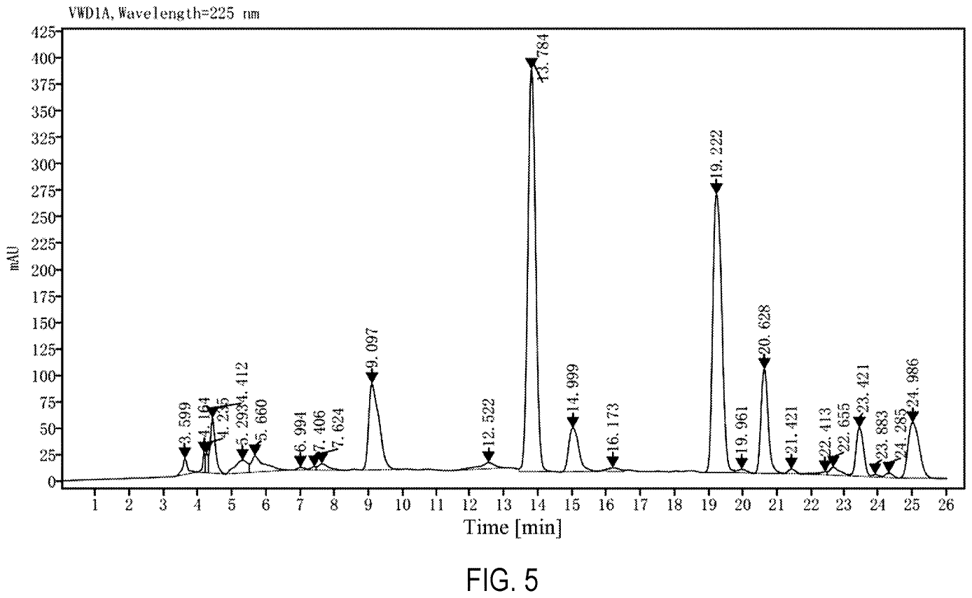

A method adopted in Comparative Example 1 is the same as that described in Example 1, except that the sZr-BTB material was not added, and a fermentation broth was directly fermented in a constant temperature shaker for shaking fermentation at 30° C. and 250 rpm for 120 h. Samples of the fermentation broth were taken every 12 h, and frozen at −20° C. in a refrigerator for preservation. A surface morphology of unloaded cells is shown in FIG. 1. It can be seen that compared with the surface morphology of unloaded cells, a surface of immobilized Yarrowia lipolytica is smooth, proving that the Zr-BTB material successfully adsorbed yeast cells. Peaks of gastrodin produced by fermentation of unloaded Yarrowia lipolytica is shown in FIG. 5, a peak of gastrodin appeared at 13.8 min with a peak area of 13.784, and a peak of a by-product p-hydroxybenzyl alcohol appeared at approximately 19.2 min with a peak area of 19.222. Yields of gastrodin inferred from peaks of different samples are shown in FIG. 3: the immobilized Yarrowia lipolytica has better catalytic activity and contributes to a higher yield of gastrodin. After 120 h of fermentation, a yield of gastrodin from fermentation of immobilized Yarrowia lipolytica reached 6 g/L, while the yield of gastrodin from fermentation of free Yarrowia lipolytica only reached 4 g/L. Ratios of gastrodin to p-hydroxybenzyl alcohol are shown in FIG. 4: the sulfonated-modified Zr-BTB inhibits the secretion of glycosidases, so a yield of p-hydroxybenzyl alcohol in a fermentation broth of immobilized Yarrowia lipolytica is much lower than a yield of p-hydroxybenzyl alcohol in a fermentation broth of free Yarrowia lipolytica.

### Example 2

In this example, an effect of an amount of ZrCl₄ in synthesized sZr-BTB ZrCl₄ on a morphology of the Zr-BTB material was explored, where the amount of ZrCl₄ affects coating of Yarrowia lipolytica by the Zr-BTB material and affects the yield of aryl glycoside compounds.

Gastrodin-producing Yarrowia lipolytica (YliGT6) was used in this example, and preparation steps of sZr-BTB are as follows:
(1) ZrCl₄, 12 mg of 1,3,5-tris(4-carboxyphenyl)benzene, and 450 mg of BA were dissolved in 3 mL of DMF, and then 0.5 mL of deionized water was added for ultrasonic dissolution. Then a resulting mixture was stirred in a water bath at 120° C. for 24 h, after cooling, and the mixture was washed with DMF for 3 times, with acetone for 3 times, with 0.1 M hydrochloric acid for once, and with deionized water for 2 times, then centrifuged at 8000 rpm for 5 min, and dried to obtain solid Zr-BTB.

Amounts of ZrCl₄ were set to 5, 10, 15, and 20 mg respectively.
(2) 80 mg of 1,2-ethanedisulfonic acid was added to 4 mL of DMF for ultrasonic dissolution, a resulting solution was added to 40 mg of Zr-BTB prepared above, after mixing uniformly, a resulting mixture was stirred in a water bath at 100° C. and 150 rpm for 20 h, and after cooling, the mixture was washed with DMF for 3 times and with acetone for 3 times, centrifuged at 8000 rpm for 5 min, and dried to obtain an sZr-BTB material.

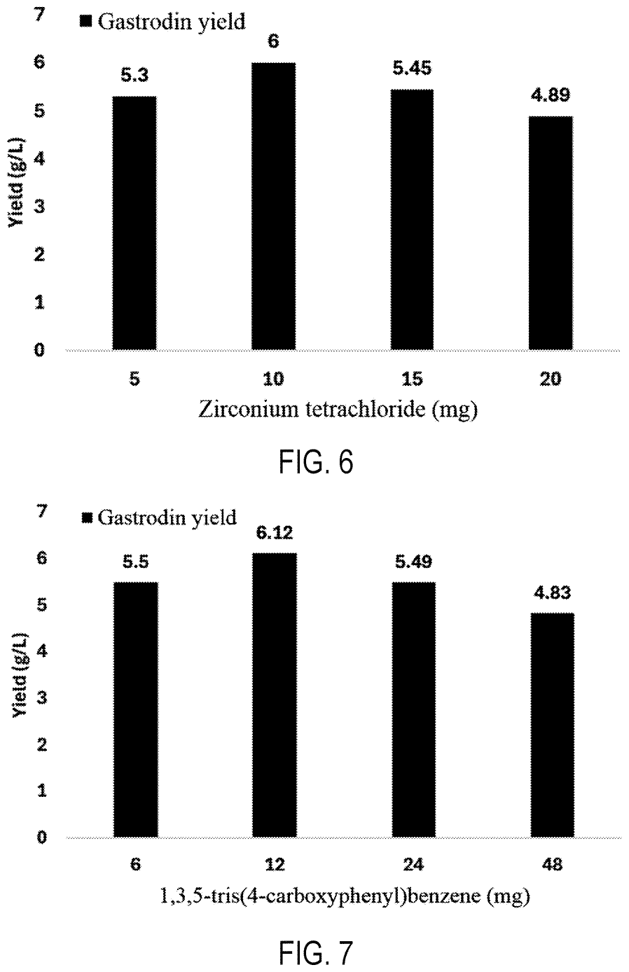

A method for preparing sZr-BTB-immobilized Yarrowia lipolytica (YliGT6) is consistent with that of Example 1. The yield of gastrodin is shown in FIG. 6, and the results show that the amount ZrCl₄ has an effect on the morphology of the produced sZr-BTB material, thereby affecting the adsorption of Yarrowia lipolytica and resulting in the change of yield. Experimental conclusions show that addition of 10 mg of ZrCl₄ is an optimal choice for preparing sZr-BTB. The yield of gastrodin was 6 g/L after 120 h of fermentation of Yarrowia lipolytica.

### Example 3

In this example, an effect of an amount of 1,3,5-tris(4-carboxyphenyl)benzene in synthesized sZr-BTB on a morphology of the Zr-BTB material was explored, where the amount of 1,3,5-tris(4-carboxyphenyl)benzene affects coating of Yarrowia lipolytica by the Zr-BTB material and affects the yield of aryl glycoside compounds. Gastrodin-producing Yarrowia lipolytica (YliGT6) was used in this example, and preparation steps of sZr-BTB are as follows:
(1) 10 mg of ZrCl₄, 1,3,5-tris(4-carboxyphenyl)benzene, and 450 mg of BA were dissolved in 3 mL of DMF, and then 0.5 mL of deionized water was added for ultrasonic dissolution. Then a resulting mixture was stirred in a water bath at 120° C. for 24 h, after cooling, and the mixture was washed with DMF for 3 times, with acetone for 3 times, with 0.1 M hydrochloric acid for once, and with deionized water for 2 times, then centrifuged at 8000 rpm for 5 min, and dried to obtain solid Zr-BTB.

Amounts of 1,3,5-tris(4-carboxyphenyl)benzene were set to 6, 12, 24, and 48 mg respectively.
(2) 80 mg of 1,2-ethanedisulfonic acid was added to 4 mL of DMF for ultrasonic dissolution, a resulting solution was added to 40 mg of Zr-BTB prepared above, after mixing uniformly, a resulting mixture was stirred in a water bath at 100° C. and 150 rpm for 20 h, and after cooling, the mixture was washed with DMF for 3 times and with acetone for 3 times, centrifuged at 8000 rpm for 5 min, and dried to obtain an sZr-BTB material.

A method for preparing sZr-BTB-immobilized Yarrowia lipolytica (YliGT6) is consistent with that of Example 1. The yield of gastrodin is shown in FIG. 7, and the results show that the amount of 1,3,5-tris(4-carboxyphenyl)benzene has an effect on the morphology of the produced sZr-BTB material, thereby affecting the adsorption of Yarrowia lipolytica and resulting in the change of yield. Experimental conclusions show that addition of 12 mg of 1,3,5-tris(4-carboxyphenyl)benzene is an optimal choice for preparing sZr-BTB. The yield of gastrodin was 6.12 g/L after 120 h of fermentation.

### Example 4

In this example, an effect of an amount of BA in synthesized sZr-BTB on a morphology of the Zr-BTB material was explored, where the amount of BA affects coating of Yarrowia lipolytica by the Zr-BTB material and affects the yield of aryl glycoside compounds. Gastrodin-producing Yarrowia lipolytica (YliGT6) was used in this example, and preparation steps of sZr-BTB are as follows:
(1) 10 mg of ZrCl₄, 12 mg of 1,3,5-tris(4-carboxyphenyl)benzene, and BA were dissolved in 3 mL of DMF, and then 0.5 mL of deionized water was added for ultrasonic dissolution. Then a resulting mixture was stirred in a water bath at 120° C. for 24 h, after cooling, and the mixture was washed with DMF for 3 times, with acetone for 3 times, with 0.1 M hydrochloric acid for once, and with deionized water for 2 times, then centrifuged at 8000 rpm for 5 min, and dried to obtain solid Zr-BTB.

Amounts of BA were set to 250, 350, 450, and 550 mg respectively.
(2) 80 mg of 1,2-ethanedisulfonic acid was added to 4 mL of DMF for ultrasonic dissolution, a resulting solution was added to 40 mg of Zr-BTB prepared above, after mixing uniformly, a resulting mixture was stirred in a water bath at 100° C. and 150 rpm for 20 h, and after cooling, the mixture was washed with DMF for 3 times and with acetone for 3 times, centrifuged at 8000 rpm for 5 min, and dried to obtain an sZr-BTB material.

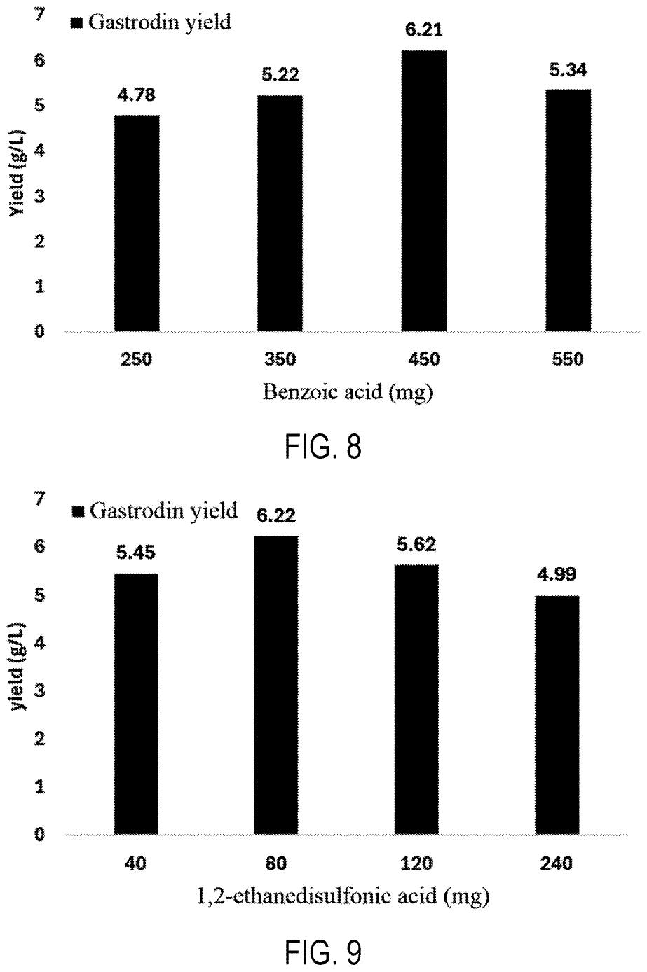

A method for preparing sZr-BTB-immobilized Yarrowia lipolytica (YliGT6) is consistent with that of Example 1. The yield of gastrodin is shown in FIG. 8, and the results show that the amount of BA has an effect on the morphology of the produced sZr-BTB material, thereby affecting the adsorption of Yarrowia lipolytica and resulting in the change of yield. Experimental conclusions show that addition of 450 mg of BA is an optimal choice for preparing sZr-BTB. The yield of gastrodin was 6.21 g/L after 120 h of fermentation of Yarrowia lipolytica.

### Example 5

In this example, an effect of an amount of 1,2-ethanedisulfonic acid providing sulfonic acid groups in synthesized sZr-BTB on adsorbing Yarrowia lipolytica was explored. Insufficient sulfonic acid groups provided may lead to the failure of long-lasting and stable adsorption of yeast by the Zr-BTB material, while excessive groups added may lead to lack of sites for the Zr-BTB material to adsorb Yarrowia lipolytica. Gastrodin-producing Yarrowia lipolytica (YliGT6) was used in this example, and preparation steps of sZr-BTB are as follows:
(1) 10 mg of ZrCl₄, 12 mg of 1,3,5-tris(4-carboxyphenyl)benzene, and 450 mg of BA were dissolved in 3 mL of DMF, and then 0.5 mL of deionized water was added for ultrasonic dissolution. A resulting mixture was transferred to a 15 mL pressure-resistant reaction tube for ultrasonic dissolution. Then a resulting mixture was stirred in a water bath at 120° C. for 24 h, after cooling, and the mixture was washed with DMF for 3 times, with acetone for 3 times, with 0.1 M hydrochloric acid for once, and with deionized water for 2 times, then centrifuged at 8000 rpm for 5 min, and dried to obtain solid Zr-BTB.
(2) 1,2-ethanedisulfonic acid was added to 4 mL of DMF for ultrasonic dissolution, a resulting solution was added to 40 mg of Zr-BTB prepared above, after mixing uniformly, a resulting mixture was stirred in a water bath at 100° C. and 150 rpm for 20 h, and after cooling, the mixture was washed with DMF for 3 times and with acetone for 3 times, centrifuged at 8000 rpm for 5 min, and dried to obtain an sZr-BTB material.

Amounts of 1,2-ethanedisulfonic acid were set to 40, 80, 120, and 240 mg respectively.

A method for preparing sZr-BTB-immobilized Yarrowia lipolytica (YliGT6) is consistent with that of Example 1. The yield of gastrodin is shown in FIG. 9, and the results show that a ratio of 1,2-ethanedisulfonic acid to Zr-BTB has an effect on the adsorption capacity of sZr-BTB. Experimental conclusions show that an optimal ratio of Zr-BTB to 1,2-ethanedisulfonic acid for preparing sZr-BTB is 1:2. When an added mass of sZr-BTB was 80 mg, the yield of gastrodin after 120 h of fermentation of Yarrowia lipolytica was 6.22 g/L.

### Example 6

In this example, an effect of a ratio between the sZr-BTB material and the microorganism Yarrowia lipolytica that are coupled on a yield of a final product was explored. Gastrodin-producing Yarrowia lipolytica (YliGT6) was used in this example, and a method for preparing sZr-BTB is consistent with that of Example 1.

The sZr-BTB-immobilized Yarrowia lipolytica (YliGT6) is prepared as follows:
(1) YliGT6 in a glycerol tube was inoculated into 3 mL of a YPD seed culture medium, and after mixing uniformly, a resulting mixture was cultured with shaking in a constant temperature shaker for shaking fermentation at 30° C. and 250 rpm for 24 h.
(2) 1.5 mL of activated Yarrowia lipolytica was inoculated into 30 mL of a fresh sugar-containing fermentation medium, and after mixing uniformly, a resulting mixture was cultured with shaking in a constant temperature shaker for shaking fermentation at 30° C. and 250 rpm for 24 h until OD₆₀₀ was approximately 0.7.
(3) sZr-BTB was added to 30 mL of a fermentation broth, and a resulting mixture was fermented in a constant temperature shaker for shaking fermentation at 30° C. and 250 rpm for 120 h. Samples of the fermentation broth were taken every 24 h, and frozen at −20° C. in a refrigerator for preservation.

Amounts of sZr-BTB were set to 0.3, 1, 5, and 10 mg respectively.
(4) The samples in the step (3) were detected by liquid chromatography.

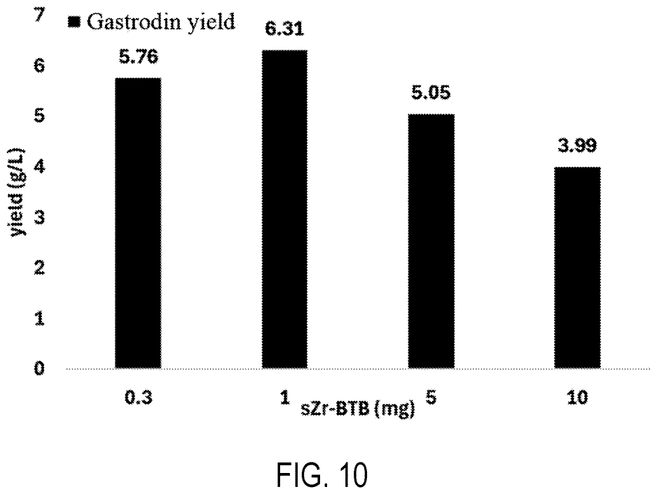

The yield of gastrodin is shown in FIG. 10, and the results show that a coupling ratio of sZr-BTB to Yarrowia lipolytica has an effect on the yield of a main product, excessive lack of the Zr-BTB material may lead to the failure to completely coat Yarrowia lipolytica, and much excess of the Zr-BTB material may result in too tight coating of Yarrowia lipolytica and reduce Yarrowia lipolytica activity. Experimental conclusions show that addition of 1 mg of sZr-BTB for 30 mL of a fermentation broth is optimal. The yield of gastrodin after 120 h of fermentation of Yarrowia lipolytica reached 6.31 g/L after adding sZr-BTB.

### Example 7

In this example, the activity and yield of Yarrowia lipolytica immobilized by sZr-BTB in Example 2 of the present disclosure at different temperatures were tested, and the activity of free Yarrowia lipolytica not immobilized by sZr-BTB was compared.

This example differs from Example 1 in that 1 mg of sulfonated-modified Zr-BTB was added to 30 mL of a fermentation broth, and a resulting mixture was fermented in a constant temperature shaker for shaking fermentation at 35° C. and 250 rpm for 120 h. Samples of the fermentation broth were taken every 24 h, and frozen at −20° C. in a refrigerator for preservation. Two additional control groups: a resulting mixture was fermented in a constant temperature shaker for shaking fermentation at 40° C. and 250 rpm for 120 h; and a resulting mixture was fermented in a constant temperature shaker for shaking fermentation at 45° C. and 250 rpm for 120 h. Subsequently, free Yarrowia lipolytica without addition of sZr-BTB was fermented at 35° C., 40° C., and 45° C. according to the same fermentation step in Example 1.

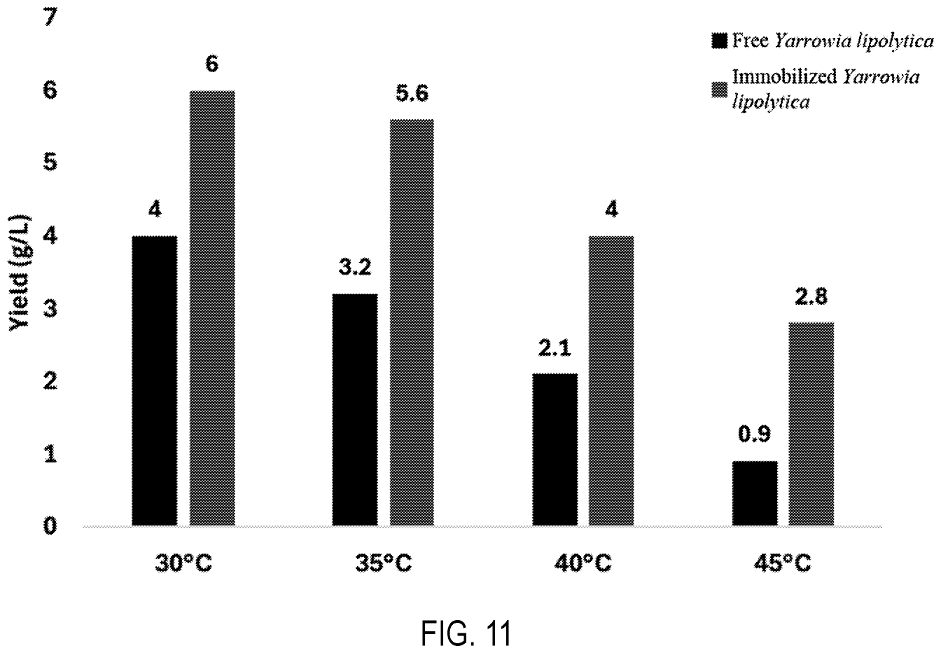

After fermentation, prepared samples were detected by high performance liquid chromatography, and the yield of gastrodin after 120 h of fermentation was calculated based on peak values, to infer the effect of different temperatures on the immobilized Yarrowia lipolytica, with results as shown in FIG. 11. It can be seen from the results that the yield of gastrodin in the fermentation broth decreased after 120 h as a fermentation temperature rose. However, the yield of gastrodin from fermentation of Yarrowia lipolytica immobilized by the Zr-BTB material at 45° C. was much higher than that of the free Yarrowia lipolytica, proving that the Zr-BTB material had a good protective effect on Yarrowia lipolytica and enhanced the stability of Yarrowia lipolytica.

### Example 8

In this example, the activity and yield of Yarrowia lipolytica immobilized by sZr-BTB in Example 1 of the present disclosure in different environments were tested, and the activity of free Yarrowia lipolytica not immobilized by sZr-BTB was compared.

This example differs from Example 1 in that 1 mg of sZr-BTB and 30 mL of methanol were added to 30 mL of a fermentation broth, and a resulting mixture was fermented in a constant temperature shaker for shaking fermentation at 30° C. and 250 rpm for 120 h. Samples of the fermentation broth were taken every 24 h, and frozen at −20° C. in a refrigerator for preservation. Two additional control groups: 1 mg of sZr-BTB and 30 mL of ethanol were added to 30 mL of a fermentation broth, a resulting mixture was fermented in a constant temperature shaker for shaking fermentation at 30° C. and 250 rpm for 120 h, and samples of the fermentation broth were taken every 24 h, and frozen at −20° C. in a refrigerator for preservation; and 1 mg of sZr-BTB and 30 mL of dimethyl sulfoxide (DMSO) were added to 30 mL of a fermentation broth, and a resulting mixture was fermented in a constant temperature shaker for shaking fermentation at 30° C. and 250 rpm for 120 h. Samples of the fermentation broth were taken every 24 h, and frozen at −20° C. in a refrigerator for preservation. Subsequently, free Yarrowia lipolytica without addition of sZr-BTB was fermented in three different organic solvents (i.e., methanol, ethanol, and DMSO) according to the same fermentation step in Example 1.

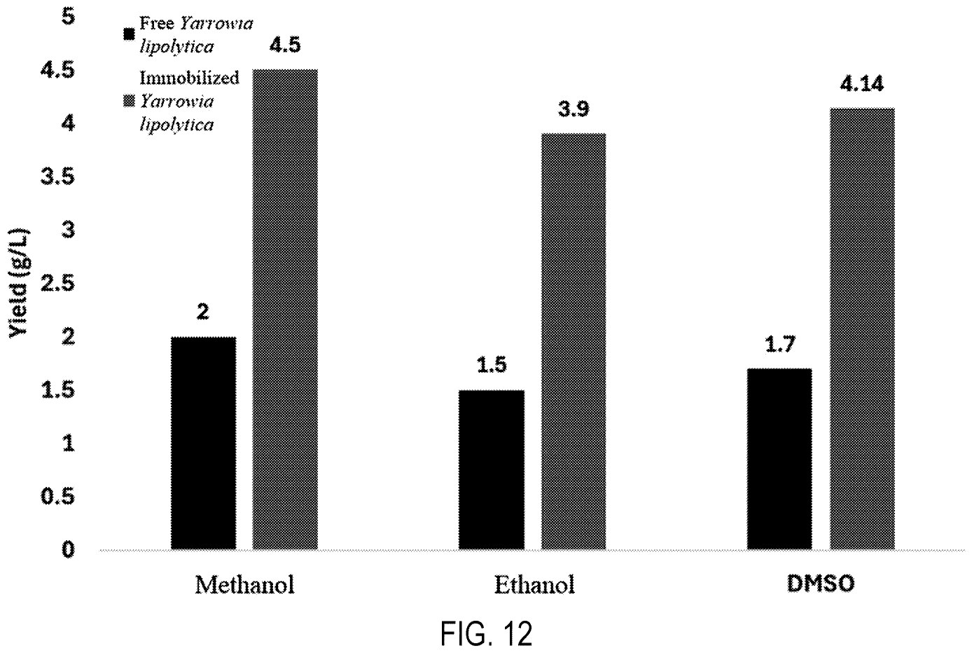

After fermentation, prepared samples were detected by high performance liquid chromatography, and the yield of gastrodin after 120 h of fermentation was calculated based on peak values, to infer the effect of organic solvents on the immobilized Yarrowia lipolytica, with results as shown in FIG. 12. It can be seen from the results that compared with free Yarrowia lipolytica, Yarrowia lipolytica immobilized by sZr-BTB showed better stability in organic solvents, and although Yarrowia lipolytica activity was still affected, the yield of gastrodin after 120 h of fermentation still accounted for 60-70% of an original yield.

### Example 9

In this example, the capability of sZr-BTB-immobilized Yarrowia lipolytica to produce other aryl glycoside compounds was explored, and the above arbutin-producing Yarrowia lipolytica was used in this example. This example differs from Example 1 in that the strain was replaced with the arbutin-producing Yarrowia lipolytica. Additionally, sZr-BTB was not added in a control group. The results show that similar to those in Example 1 and Comparative Example 1, the sZr-BTB material in this example enhanced the catalytic activity of the arbutin-producing Yarrowia lipolytica, inhibited the secretion of glycosidases, and reduced the production of hydroquinone.

In summary, the sulfonated-modified Zr-BTB (sZr-BTB) material prepared in the present disclosure plays an important role in yield increase of aryl glycoside compounds produced by fermentation of immobilized Yarrowia lipolytica. Free Yarrowia lipolytica extracellularly secretes small-molecule products such as glycosidases during fermentation, resulting in the decomposition of the main product aryl glycoside compounds by glycosidase (Comparative Example 1). Additionally, a lot of organic acids are produced during fermentation of Yarrowia lipolytica, resulting in that pH values of a fermentation broth drop to 2-3. A strongly acidic environment harms yeast and even causes yeast death, and Compared with Comparative Example 1, the present disclosure still maintains excellent activity in a strongly acidic environment. The sZr-BTB prepared in the present disclosure competes with groups on a yeast cell surface for hydrogen ions in a highly acidic fermentation broth, thereby maintaining the long-lasting and stable physical adsorption of yeast cells by the Zr-BTB material and protecting Yarrowia lipolytica cells. Moreover, adsorption and embedding of yeast cells prevent the extracellular secretion of glycosidases from yeast, inhibit the decomposition of the main product by enzymes, and increase the yield from microbial fermentation.

What is claimed is:

1. A sulfonated-modified metal-organic framework material, wherein the sulfonated-modified metal-organic framework material is prepared through a reaction with 1,2-ethanedisulfonic acid for sulfonation modification wherein a zirconium (Zr)-based MOF material Zr-BTB serves as a precursor.

2. A method for preparing the sulfonated-modified metal-organic framework material according to claim 1, comprising the following steps:
(1) preparing a metal-organic framework material Zr-BTB: dissolving 1,3,5-tris(4-carboxyphenyl)benzene (H₃BTB), benzoic acid (BA), and zirconium tetrachloride (ZrCl₄) in an organic solvent;
(2) adding deionized water to the organic solvent, performing ultrasonic mixing for a high-temperature reaction, and washing after cooling, and centrifuging and drying to obtain the Zr-BTB material as a precursor; and
(3) adding 1,2-ethanedisulfonic acid to the prepared Zr-BTB material as the precursor, centrifuging a resulting mixture after a high-temperature reaction, collecting a precipitate and washing with deionized water to obtain a sulfonated-modified Zr-BTB material, i.e., the sulfonated-modified metal-organic framework material.

3. The preparation method according to claim 2, wherein in the step (1), a mass ratio of H₃BTB to BA to ZrCl₄ is 10-13:440-460:9-12.

4. The preparation method according to claim 2, wherein in the step (2), the high-temperature reaction is performed at 100-130° C. for 20-24 h, and the centrifuging is performed at 8000-12000 rpm for 5-10 min.

5. The preparation method according to claim 2, wherein in the step (3), 1,2-ethanedisulfonic acid is dissolved in the organic solvent, a mass ratio of 1,2-ethanedisulfonic acid to Zr-BTB is 1:1-6:1, the high-temperature reaction is performed at 90-120° C. for 15-20 h, and the centrifuging is performed at 8000-12000 rpm for 5-10 min.

6. An application of the sulfonated-modified metal-organic framework material according to claim 1 in production of aryl glycoside compounds by fermentation of Yarrowia lipolytica.

7. The application according to claim 6, wherein a process of the application comprises the following steps:
(1) inoculating Yarrowia lipolytica into a seed culture medium for seed culture;
(2) inoculating a seed culture solution into a fermentation medium for fermentation culture; and
(3) adding the sulfonated-modified metal-organic framework material into a fermentation broth, and continuing fermentation to achieve yield increase of a target product, i.e., aryl glycoside compounds.

8. The application according to claim 7, wherein in the step (1), the Yarrowia lipolytica is cultured in a yeast extract peptone dextrose (YPD) seed culture medium for 24-36 h; and in the step (2), culture is further performed in the fermentation medium for 24-36 h.

9. The application according to claim 7, wherein in the step (3), the sulfonated-modified metal-organic framework material is added to the fermentation broth, specifically, 0.5-1.5 mg of the sulfonated-modified Zr-BTB material is added per 20-40 mL of the fermentation broth, immobilization starts in a logarithmic growth phase, and fermentation is continued for 100-120 h to achieve the yield increase of the target product, i.e., aryl glycoside compounds.

10. The application according to claim 6, wherein the aryl glycoside compounds preferably comprise one or more of gastrodin, arbutin, resveratrol, naringin, scutellarin and isovitexin.


SEQUENCE LISTING (3 sequences)

SEQ ID NO: 1 - DNA, 1410 bp, synthetic construct
  source 1..1410: mol_type = other DNA
gaacacacccctcacattgctatggtgcccactcctggcatgggccacctgattcctctggtcgagttcgccaagcgactggtgctgcgacacaacttcggcgtgaccttcatcatccccaccgacggacccctgcctaaggctcagaagtctttcctggacgccctgcctgccggcgtgaactacgtgctgctgcctcctgtgtctttcgacgatctgcccgccgacgtgcgaatcgagactcgaatctgcctgaccatcactcgatctctgcccttcgtgcgagatgccgtcaagaccctgctggccaccaccaagctggctgccctggtggtggacctgttcggcaccgacgccttcgacgtggccatcgagttcaaggtgtctccctacatcttctaccccaccaccgccatgtgcctgtctctgttcttccatctgcctaagctggaccagatggtgtcttgcgagtaccgagatgtgcccgagcctctgcagatccccggctgcatccccatccacggcaaggactttctggaccccgctcaggaccgaaagaacgacgcctacaagtgcctgctgcaccaggccaagcgataccgactggccgagggcatcatggtgaacaccttcaacgacctcgagcctggacctctgaaggccctgcaagaagaggaccagggcaagcctcctgtctaccccattggacccctgatccgagccgactcttcgtctaaggtggacgactgcgagtgcctgaagtggctggacgaccagcctcgaggatctgtgctgttcatctctttcggctctggcggagccgtgtctcacaaccagttcatcgagctggccctgggcctcgagatgtctgagcagcgattcctgtgggtcgtgcgatctcccaacgacaagatcgccaacgccacctacttctctatccagaaccagaacgatgccctggcctacctgcctgagggcttcctcgagcgaactaagggccgatgcctgctggtgccctcttgggctccccagaccgagatcctgtctcacggctctaccggcggctttctgacccactgcggctggaactctatcctcgagtctgtggtgaacggcgtgcccctgatcgcctggcctctgtacgccgagcaaaagatgaacgccgtgatgctgaccgagggactgaaggtggctctgcgacccaaggccggcgagaacggcctgatcggccgagtcgagattgccaacgctgtgaagggcctgatggaaggcgaggaaggcaagaagttccgatctaccatgaaggacctgaaggacgccgcctctcgagccctgtctgacgacggatcttccaccaaggctctcgccgagctggcttgcaagtgggagaacaagatctcttctacctaa

SEQ ID NO: 2 - DNA, 495 bp, synthetic construct
  source 1..495: mol_type = other DNA
tctcaccctgccctgacacagctgagagccctccggtatttcaaagagatccccgcccttgatccccagctgctggactggctgctgctccaggattcggttactaagcggtttgagcaacaaggcaaaaccgttagcgtcaccatgatccgagaaggtttcgtggagcaaaatgaaattcctgaggaactgcccctcctgcctaaagaatcgcggtactggctcagagagatcctgctctgcgctgatggcgagccttggctcgctggccgaaccgtggtgcctgtgtccacactttctggacccgaacttgctctccaaaaacttggcaaaacacctctcggtagatacctttttacttcttcgactctcacacgggactttattgaaatcggacgggatgctggactgtggggacgtcgatctcgtctgcgactctcgggcaaacccctcctccttactgaactcttccttcccgcatctcccctttattaa

SEQ ID NO: 3 - DNA, 1437 bp, synthetic construct
  source 1..1437: mol_type = other DNA
gccgtgcaggctccctctaagacctacggtttccagaaggcccctattcagcttactttcgtcgttgtgggcgctggtcttggcggtgtcgccgcttccatttgcctgcgactggctggtcaccgagtcatcctgctggaagccgctactgaattaggagaggtcggtgctggtattcagatccctcctccctctactaagattctgaaggctatcggagttcttgacgccgttgacaaggtgtctatccaccctcacgacatccttgttaagaagtacaagggtgagctgctgtctactcagaaccttgtgccttacgttcttgagaagtacgacggcatgtacctgcacatccaccgagccgactaccacaaggttctcgtggatcgagctgaggagcttggcgttgagattcacactaacagtagagtcgtggacattgacttcgagaaggccaccgtcactaccgccactggaaagcagtactccggtgacgtcatcgtgggatacgacggagtgcgatctcagacccgagccctgctcactggtgactcttccggcgcttacgacaccggagaccttgcttaccgagcccttatcaaggttgaggacatgaagaaggttcccggtctggagaagttctacgccaaccctaacatcaacttctggtggggtcccactatgcacattgttatgtacttcctgcacgagggagagatttgcaacgtcgttgctctgtgccccgacactctgcccaagggcgtcctgaagcaggacgcctcccaggaggaactactggaccttgttaagggatgggaccaggacctcaccactgttttcaagctgattacttctgtgtccaagtggcgactacaagactcgcgtgagcttaagacttgggttaactctaagaccggaaacttcattattcttggtgacgcctctcactctactctgccttaccttgcctccggcgcctctcaggccgtggaggacggtgctgttctcgctggtcttttctccaagattgagcttcgagaccagatcccccagctcttacagatgactgagaacctgcgaaagtggcgatcttcccaggttgtgcgaggatctcaccagtgccaggacatttaccacctgcccgacggtgagcttcaggagattcgagactcctacctgtacgacaagcagcctgaactcggatgccccaaccgattcgctgaccccgttttccaggacttcctttggggatacaacgctttcgacgaggttgagcgagcttggaaggagttcaaggctggcggaaaccccacttacacctaccctaacctttacaagcctaagtcttccggagagaaggacgtgtccggcggcggcgccgctgctaccctcgccgccggcaacacccccgccgctcctctgtccgcttccggctag